(19) United States
(12) Patent Application Publication
(10) Pub. No.: US 2005/0037952 A1
(43) Pub. Date: Feb. 17, 2005

(54) Effect of bone morphogenetic proteins on engineered cartilage

(76) Inventors: Lisa E. Freed, Lexington, MA (US); Keith J. Gooch, Media, PA (US); Donald L. Courter, Seattle, WA (US); Torsten Blunk, Pentling (DE); Alisha L. Sieminski, Jamaica Plain, MA (US); Gordana V. Vunjak-Novakovic, Belmont, MA (US)

Correspondence Address: Valarie B. Rosen;Choate, Hall & Stewart, Exchange Place, 53 State Street, Boston, MA, 02109, US

(21) Appl. No.: 10/457,248

(22) Filed: Jun. 9, 2003

Related U.S. Application Data
(63) Continuation of application No. 10/262,867, filed on Oct. 3, 2002, now abandoned.
(60) Provisional application No. 60/327,141, filed on Oct. 4, 2001.

(51) Int. Cl. A61K 38/18

(52) U.S. Cl. 514/12

(57) ABSTRACT

Compositions and methods comprising at least one active agent chosen from TGF-P superfamily proteins, including BMPs, and GDFs for increasing the growth rate or modulating the development of engineered cartilage are disclosed. The compositions are useful in the treatment of osteoarthritis, cartilage defects, and in related tissue repair.

### RELATED APPLICATION

This application claims the benefit of priority of U.S. Provisional Application No. 60/327,141, filed Oct. 4, 2001.

### FIELD OF THE INVENTION

The search for the molecule or molecules responsible for formation of bone, cartilage, tendon, and other tissues present in bone and other tissue extracts has led to the discovery of a set of molecules called Bone Morphogenetic Proteins (BMPs). The structures of several proteins, designated BMP-1 through BMP-16, have previously been elucidated. The unique inductive activities of these proteins, along with their presence in bone, suggest that they are important regulators of bone repair processes, and may be involved in the normal maintenance of bone tissue. It has now been found, as described herein, that members of this subfamily are effective for increasing the growth rate or the modulation of engineered cartilage, and thus are useful for the treatment of diseases or defects of cartilaginous tissue.

### SUMMARY OF THE INVENTION

The present invention relates to a composition for increasing the growth rate or modulating the development of engineered cartilage comprising at least one active agent chosen from TGF-β superfamily proteins, including BMPs and GDFs, in combination with at least one biomaterial scaffold as a carrier. In an embodiment, the composition comprises at least one active agent chosen from BMP-2, BMP-12, and BMP-13.

The method of the present invention comprises adding a composition comprising at least one active agent chosen from the TGF-β superfamily proteins, including BMPs and GDFs, in combination with at least one biomaterial scaffold, to engineered cartilage in an amount effective for increasing the growth rate or modulating the development of the engineered cartilage.

The present invention also includes methods for cartilaginous tissue healing and tissue repair, for treating osteoarthritis, or other cartilage defects, and for inducing cartilaginous tissue formation in a patient in need of same, comprising administering to said patient an effective amount of the above composition comprising the BMP modulated engineered cartilage.

A further aspect of the invention is a therapeutic method and composition for repairing cartilaginous tissue, for repairing cartilage as well as treating arthritis and other conditions related to arthritis defects. Such compositions comprise a therapeutically effective amount of BMP modulated engineered cartilage tissue.

### BRIEF DESCRIPTION OF THE DRAWINGS

FIG. 1 sets forth wet weight of engineered cartilage after 4 weeks of culture in control medium (no fill) or medium supplemented with 1 ng/ml, 10 ng/ml, or 1000 ng/ml of the indicated BMP or 50 ng/ml of IGF-I. Each bar represents the standard error of the mean except for control, which represents 12 constructs. A star directly above an error bar represents that the average value is significantly different from control values (p<0.05). A horizontal bar with a star represents that the two treatments are significantly different (p<0.05)

FIG. 2 sets forth data regarding cells as a percentage of wet weight of engineered cartilage after 4 weeks of culture in control medium (no fill) or medium supplemented with 1 ng/ml, 10 ng/ml, or 1000 ng/ml of the indicated BMP or 50 ng/ml of IGF-I. Total cell number was calculated from measured DNA content and normalized by the construct wet weight. Each bar represents the average values for 3 constructs and each error bar represents the standard error of the mean except for control, which represents twelve constructs.

FIG. 3 sets forth data regarding GAG as a percentage of wet weight of engineered cartilage after 4 weeks of culture in control medium (no fill) or medium supplemented with 1 ng/ml, 10 ng/ml, or 1000 ng/ml of the indicated BMP or 50 ng/ml of IGF-I. Each bar represents the average values for 3 constructs and each error bar represents the standard error of the mean except for control, which represents twelve constructs.

FIG. 4 sets forth data regarding collagen as a percentage of wet weight of engineered cartilage after 4 weeks of culture in control medium (no fill) or medium supplemented with 1 ng/ml, 10 ng/ml, or 1000 ng/ml of the indicated BMP or 50 ng/ml of IGF-I. Each bar represents the average values for 3 constructs and each error bar represents the standard error of the mean except for control, which represents twelve constructs.

FIG. 5 sets forth data regarding correlation of the percent wet weight of (A) GAG and (B) collagen with total construct wet weight after 4 weeks of culture in control medium or medium supplemented with 1 or 10 ng/ml of BMP-2, BMP-12 or BMP-13 (0), 50 ng/ml of IGF-I(X), or 100 ng/ml of BMP-2 ( ), BMP-12( ), or BMP-13 ( ). The lines represent linear regression fits through data for each construct (i.e. n=42).

### DETAILED DESCRIPTION OF THE INVENTION

Composition

The present invention relates to a composition for increasing the growth rate or modulating the development of engineered cartilage comprising at least one active agent. Engineered cartilage is understood to mean cartilage that is prepared by seeding isolated cells onto a three-dimensional biodegradable, biomaterial scaffold followed by culturing the cells in a laboratory or implanting them in vivo. See Pei, M., “Bioreactors mediate the effectiveness of tissue engineering scaffolds,” The FASEB Journal, published online Aug. 7, 2002.

The present invention also relates to a composition for use with sutures as well as cartilage allografts and autographs for promoting cartilage growth.

The at least one active agent administered to the engineered cartilage is chosen from proteins known as Transforming Growth Factor-Beta (TGF-β) superfamily proteins, including Bone Morphogenetic Proteins (BMPs) and Growth and Differentiation Factors (GDFs), as well as other proteins, as described more fully herein. Osteogenic proteins, useful in the present invention include, for example, BMP proteins including BMP-1, BMP-2, BMP-3, BMP-4, BMP-5, BMP-6 and BMP-7, disclosed for instance in U.S. Pat. Nos. 5,108,922; 5,013,649; 5,116,738; 5,106,748; 5,187,076; and 5,141,905; BMP-8, disclosed in PCT publication WO 91/18098; BMP-9, disclosed in PCT publication WO 93/00432; BMP-10, disclosed in PCT application WO 94/26893; and BMP-11, disclosed in PCT application WO 94/26892. BMP-12 related proteins are a subset of the BMP/TGF-βg-1 family of proteins, including BMP-12 and BMP-13, which have previously been shown to have tendon/ligament-like tissue inducing ability, and which are encoded by DNA sequences which have been cloned and identified. This subfamily also includes MP52, which is described in WO 93/16099. The BMP-12 related family of proteins, the DNA sequences encoding them, vectors, host cells, compositions and methods of making the proteins have all been extensively described in WO 95/16035, as well as U.S. Pat. Nos. 5,658,882; and 6,027,919. Additionally, BMP-15, disclosed in PCT application WO 96/36710 or BMP-16, disclosed in U.S. Pat. No. 5,965,403, may be suitable in the present invention. In an embodiment of the present invention, the composition comprises at least one active agent chosen from BMP-2, BMP-12, and BMP-13.

Other proteins and the DNA sequences encoding them which are capable of increasing the growth rate or otherwise modulating engineered cartilage tissue may also be utilized.

Proteins which maybe useful include those encoding Vgr-2, and any of the growth and differentiation factors (GDFs), including, for example, those described in PCT applications WO 94/15965; WO 94/15949; WO 95/01801; WO 95/01802; WO 94/21681; and WO 94/15966. Also useful in the present invention may be a bone-formation inducing protein (BIP), disclosed in WO 94/01557.

Other proteins which may be useful, include therapeutically useful agents including growth factors such as epidermal growth factor (EGF); fibroblast growth factor (FGF); transforming growth factor (TGF-α and TGF-β); hedgehog proteins such as sonic, Indian, and desert hedgehog; parathyroid hormone and parathyroid hormone related peptides, cadherins, activins, inhibins, and IGF; FSH; frizzled, frzb orfrazzled proteins; PDGF and other endothelial growth factors; BMP binding proteins such as chordin and fetuin; estrogen and other steroids as well as truncated versions thereof; and transcription factors such as wnt proteins, mad genes and cbfa.

The composition may include an appropriate biomaterial scaffold as a carrier. For instance, the biomaterial scaffold may support the composition or provide a surface for cartilaginous tissue growth or formation and/or other tissue formation.

The choice of a biomaterial scaffold is based on biocompatibility, biodegradability, mechanical properties, cosmetic appearance, and interface properties. The particular application of the composition will define the appropriate formulation of the biomaterial scaffold. For example, biomaterials used in engineered cartilage studies have varied widely with respect to scaffold material, SM (e.g., synthetic, semi-synthetic, and naturally occurring polymers), scaffold structure (e.g., hydrogels, fibrous meshes, and sponges), and with respect to mechanical properties and degradation rate. Potential biomaterial scaffolds for the composition may be biodegradable or non-biodegradable and of a known chemical structure. Biomaterial scaffolds comprise pure proteins or extracellular matrix components. Examples of these biomaterials include gels of fibrin or collagen, such as collagen in an injectable form, and meshes or sponges of collagen, such as HELISTAT® (Integra LifeSciences, Plainsboro, N.J.), or cross-linked and/or derivatized hyaluronic acid. Biodegradable materials, such as cellulose films, or surgical meshes, may also serve as biomaterial scaffolds. The biomaterial scaffold could be sutured into an injury site, or wrapped around the cartilage.

Another preferred class of biomaterial scaffolds are polymeric matrices, including polymers of poly(lactic acid), poly(glycolic acid) and copolymers of lactic acid and glycolic acid. These matrices may be in the form of a sponge, or in the form of porous particles. Suitable polymer matrices are described, for example, in WO 93/00050.

The composition of the present invention is a physiologically acceptable composition. The preparation and formulation of such physiologically acceptable compositions, having due regard to pH, isotonicity, stability and the like, is within the skill of the art. The compositions are also presently valuable for veterinary applications due to the lack of species specificity in TGF-β family proteins. Particularly, domestic animals and thoroughbred horses, in addition to humans, are desired patients for treatment with the composition of the present invention.

Applications

The composition of the present invention has application in the healing of cartilage, for example articular cartilage tears, deformities and other cartilage defects in humans and other animals. For example, a composition of the invention may be used to improve fixation of cartilage to bone or other tissues, and to repair defects to cartilage tissue. The composition of the invention may also contribute to the repair of congenital, trauma induced, or other cartilage defects of other origin, and are also useful in surgery for attachment or repair of cartilage. The composition of the invention may also be useful in the treatment of arthritis and other cartilage defects. The composition of the present invention can also be used in other indications wherein it is desirable to heal or regenerate cartilage tissue. Such indications include, without limitation, regeneration or repair of injuries to the articular cartilage. The composition of the present invention may provide an environment to attract cartilage-forming cells, stimulate growth of cartilage-forming cells, or induce differentiation of progenitors of cartilage-forming cells.

By cartilaginous tissue, it is meant chondrocytes, and tissue which is formed by chondrocytes, which demonstrate the histological and compositional characteristics of cartilage. This tissue includes, for example, tissue which exhibits the marker proteins characteristic of cartilage and/or chondrocytes, which are described further herein, such as aggrecan, type II collagen, and proteoglycan core protein.

Method

In the method of the present invention, the composition described above, comprising, for example, at least one BMP protein or at least one BMP related protein is added to engineered cartilage. In an embodiment, the composition comprises at least one active agent chosen from BMP-2, BMP-12, and BMP-13. See Gooch, K., “Bone Morphogentic Proteins-2, -12, and -13 Modulate in vitro Development of Engineered Cartilage,” Tissue Engineering, 8(4): 591-601 (2002). The at least one active agent may increase the growth rate or otherwise modulate the development of the engineered cartilage. The ability of the protein, for example, a BMP, to increase the growth rate of cartilage decreases the time required to generate constructs with a given size or in a given time to produce a larger construct from a given number of harvested cells.

The modulated engineered cartilage composition may be useful for the induction and maintenance of cartilaginous tissue at a site in need of cartilage repair, such as an articular cartilage defect.

The therapeutic method includes administering the composition of the invention in accordance with methods known to those skilled in the art. Administration may be locally as an implant or device. When administered, the therapeutic composition for use in this invention is, of course, in a pyrogen-free, physiologically acceptable form. Further, the composition may desirably be encapsulated for delivery to the site of tissue damage. In addition, the composition of the present invention may be used in conjunction with presently available treatments for cartilage injuries, such as suture (e.g., vicryl sutures or surgical gut sutures, Ethicon Inc., Somerville, N.J.), cartilage allograft, or autograft, in order to enhance or accelerate the healing potential of the suture or graft. For example, the suture, allograft, or autograft may be soaked in the composition of the present invention prior to implantation. It may also be possible to incorporate the protein or composition of the invention onto suture materials, for example, by freeze-drying.

In another embodiment, the method may entail administration of a heterodimeric protein in which one of the monomers is a cartilaginous tissue modulating polypeptide, such as BMP-2, BMP-12, or BMP-13, and the second monomer is a member of the TGF-β superfamily of growth factors.

The regimen will be determined by the attending physician considering various factors which modify the action of the composition, e.g., amount of cartilaginous tissue desired to be formed, the site of cartilaginous tissue damage, the condition of the damaged cartilaginous tissue, the size of a wound, type of damaged tissue, the patient's age, sex, and diet, the severity of any infection, time of administration, and other clinical factors.

Progress can be monitored by periodic assessment of cartilaginous tissue formation, or cartilaginous tissue growth and/or repair. The progress can be monitored by methods known in the art, for example, X-rays, arthroscopy, histomorphometric determinations and tetracycline labeling.

### EXAMPLES

The following examples demonstrate the ability of the composition of the invention to increase the growth rate or otherwise modulate engineered cartilage. Although the examples demonstrate the invention with respect to BMP-2, 12 and 13 as the cartilage-modulating protein, with minor modifications within the skill of the art, the same results may be attainable with other proteins.

### Example 1

### Isolation of Chondrocytes

Primary chondrocytes were isolated from full-thickness bovine calf articular cartilage by digestion with type II collagenase (Worthington, Freehold N.J.) and resuspended in culture medium high glucose Dulbecco's modified Eagle's medium (DMEM) (Gibco, Grand Island, N.Y.) containing 4.5 g/l glucose, 584 mg/l glutamine, 10% fetal bovine serum (FBS) (Gibco), 50 U/ml penicillin, 50 μg/ml streptomycin, 10 mM N-2-hydroxyethylpiperazine N′-2-ethanesulfonic acid (HEPES), and 0.1 mM nonessential amino acids. 0.4 mM proline and 50 μg/ml ascorbic acid as described in J Biomed Mater Res 27:11-23 (1993) were then added. Chondrocytes were harvested from a total of 3 or more knee joints from 2-4 week old bovine calves within 8 hours.

### Example 2

### Preparation of Scaffolds, Cell Seeding and Culturing

A polyglycolic acid (PGA) scaffold (Albany International Mansfield, Mass.) is described in Biotechnology 12689-693 (1994). PGA was extruded into 13 μm diameter fibers, processed to form a 97% porous non-woven mesh with a bulk density of 62 mg/cm³, die punched into discs 5 mm in diameter by 2 mm thick, and sterilized with ethylene oxide. Cell seeding of PGA scaffolds is described in Biotech Prog 14 193-202 (1998). Scaffolds were threaded onto 4-inch long, 22-gauge needles (Metropolotan Hospital Supply, Cambridge, Mass.) and held in place with 3 mm-long segments of silicone tubing (#13 Cole Palmer Niles, Ill.). Three needles with two scaffolds apiece were inserted into a silicone stopper which was in turn placed into the mouth of a spinner flask containing a magnetic stir bar (Bellco Vineland, N.J.) and 120 ml of culture medium. The side arms of the flasks were loosened to permit gas exchange and the flasks were placed on a magnetic stir plate at 80 rpm in a humidified 37° 5% CO₂ incubator. After 16 hours, spinner flasks were inoculated with a suspension of five million freshly isolated chondrocytes per scaffold and mixed at 80 rpm. Under these conditions, the seeding of chondrocytes onto PGA scaffolds was previously shown to be reproducible, essentially 100% efficient, and to yield a high initial cell density (Biotec Prog 14: 193-202 (1998)). After two additional days, cell-PGA constructs were transferred into 6-well plates (one construct and 6 ml of medium per well), and cultured on an orbital shaker at 50 rpm. Medium supplemented with appropriate biological factors BMP-2, BMP-12, BMP-13 (Genetics Institute, Cambridge Mass.) or IGF-I (R&D Systems Minneapolis, Minn.) was completely exchanged three times per week for 4 weeks.

### Example 3

### Analysis of Engineered Tissues

Engineered tissues (n=3 for each treatment and n=12 for control conditions) were weighed, frozen, lyophilized, and digested with proteinase K at 60° C. for 16 hours (1 mg/ml proteinase K in buffered solution, 1 ml enzyme solution for up to 20 mg dry weight sample) (J. Clin Invest 93: 1722-1732 (1994)). Sulfated GAG content was determined spectrophotometrically at 525 nm after reaction with dimethylmethylene blue dye (Aldrich Milwaukee, Wis.) using bovine chondroitin sulfate as standard (Biochim Biophys Acta 883: 173-177 (1986)). Hydroxyproline content was determined spectrophotometrically after acid hydrolysis and reaction with p-dimethylaminobenzaldehyde (Fisher, Paris, Ky.) and chlotamine-T (Mallinckrodt, Fairlawn, N.J.) (Archiv Biochem Biophys 93:440-447 (1961)) and the amount of total collagen was calculated using a 1:10 ratio of hydroxyproline to collagen (J. Clin Invest 93:1722-1732 (1994)). The number and mass of cells per cell-polymer construct was assessed from the DNA content using a spectrofluorometer and conversion factors of 7.7 pg DNA per chondrocyte (Anal Biochem 174: 168-176 (1988)) and 10(−10)g per chondrocyte (Biotechnology 12:689-693 (1994)). Undegraded polymer was assumed not to account for a significant fraction of the wet weight of a 4-week construct (approximately 1 to 2%). Samples for histological analyses were fixed in 2% glutaraldehyde in phosphate buffered saline (PBS)(Gibco) for 15 minutes, then in 10% neutral formalin, and then embedded in paraffin and cross-sectioned (5 μm thick). Deparaffinized sections were stained with hematoxylin and eosin (H&E) or safranin-O to visualize GAGs (glycosaminoglycans). A light microscope fitted with an eyepiece reticle was used to measure the distance from the construct surface to: (i) the first region positively stained for GAG with safranin-O (hereby referred to as the lower limit of capsule thickness) and (ii) the first region containing round cells in lacunae (hereby referred to as the upper limit of the capsule thickness), in 12 randomly selected regions of representative histological sections from each group. Statistical significance (P<0.05) was assessed using one-way analysis of variance (ANOVA) (a=0.05) followed by Scheffe's multiple comparisons procedure.

### Example 4

### Effect of Protein Compositions on Engineered Tissue

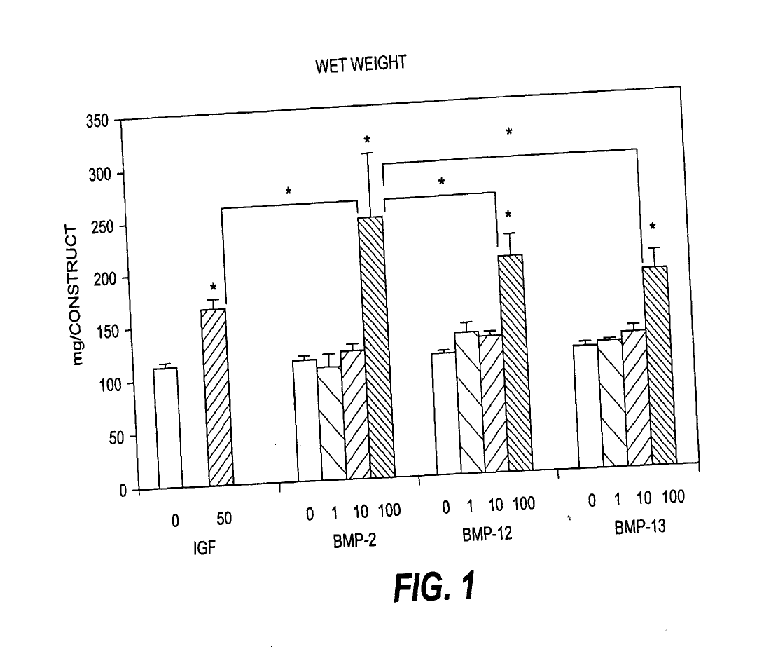

Under all conditions investigated, the cell-polymer constructs developed over 4 weeks of in vitro cultivation into cartilaginous tissues that macroscopically resembled native cartilage. Consistent with previous observations, safranin-O stained histological sections of constructs grown in control cell culture medium revealed two relatively distinct regions: an inner region consisting of round chondrocytes in individual lacunae and a GAG-rich ECM, and an outer capsule consisting of elongated cells and GAG-deficient ECM. The total thickness of the constructs was approximately 3 to 6 mm. In control medium, capsule thickness had a lower and upper limit of 111±12 and 460±114 μm, respectively. With the addition of 100 ng/ml of BMP-2, BMP-12, or BMP-13, capsule thickness lower limits were respectively decreased to 51±14, 96±8 and 46±12 μm, and upper limits were respectively decreased to 120±25, 240±30, 300±36 μm. In contrast, the addition of 50 ng/ml of IGF-I did not affect the capsule thickness (lower and upper limits were 120±15 and 550±46 μm, respectively, FIG. 1). In the presence of 100 ng/ml of BMP-2, hypertrophic chondrocytes were observed at depths of approximately 50 μm to 1-2 mm from the construct surface, whereas chondrocytes more than 1-2 mm from the surface were much less affected.

Addition of 100 ng/ml of BMP-2, BMP-12, or BMP-13 increased weight of the constructs (FIG. 1: 121%, 80%, 62% increase over control values of 111.4±2.9 mg, respectively). Lower concentrations of the BMPs did not result in a statistically significant increase of wet weights. Conditions that increased the total mass of the construct consistently resulted in a corresponding increase in the mass of GAG and collagen as well as the number of cells. To examine the effects of the BMPs on the composition of the constructs composed primarily of cells, GAG, and collagen, the total mass of each of these components was normalized by the total wet weight to give a weight percent.

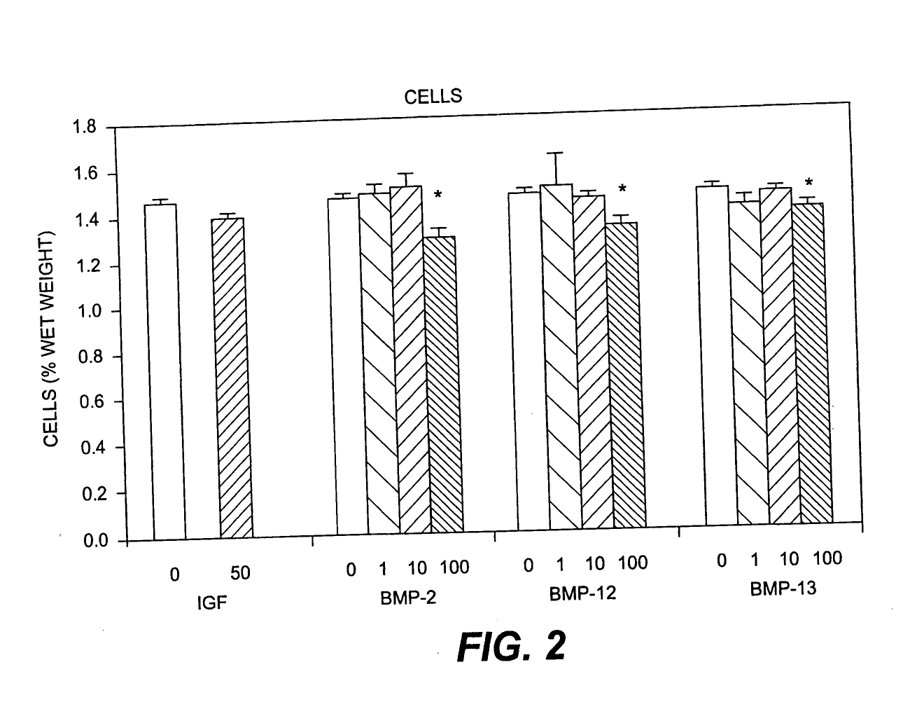

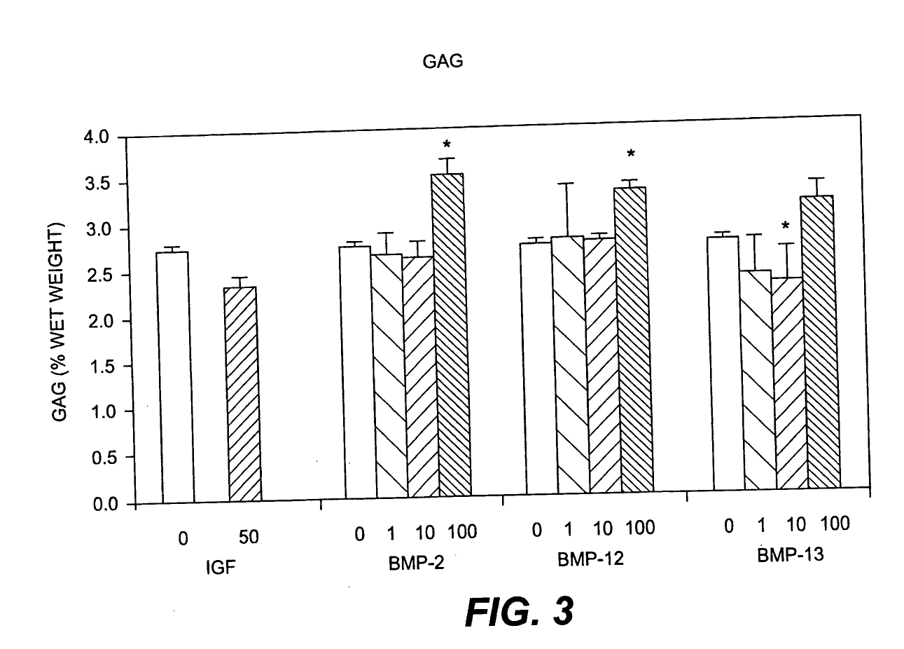

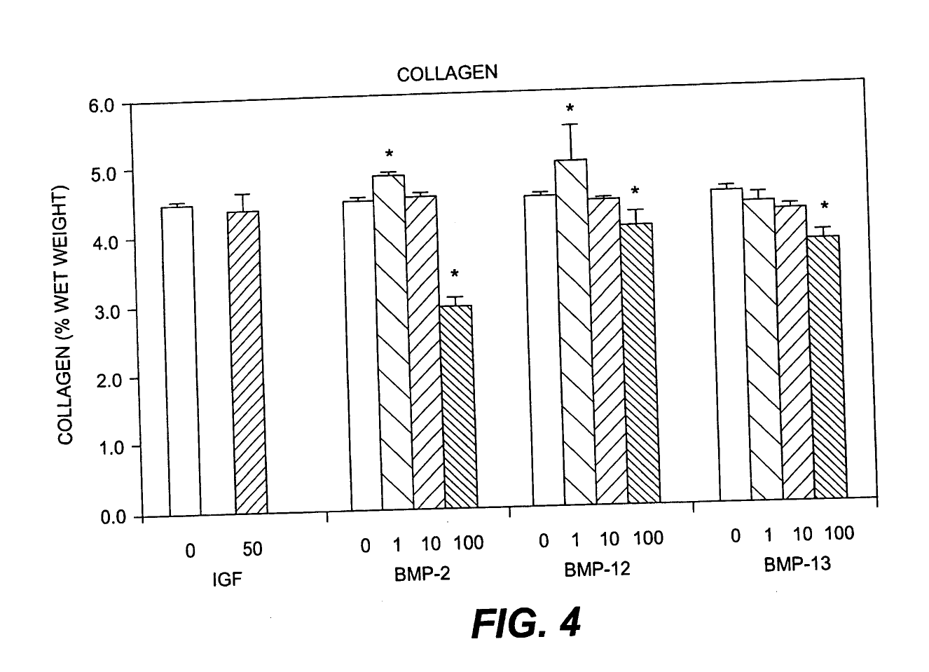

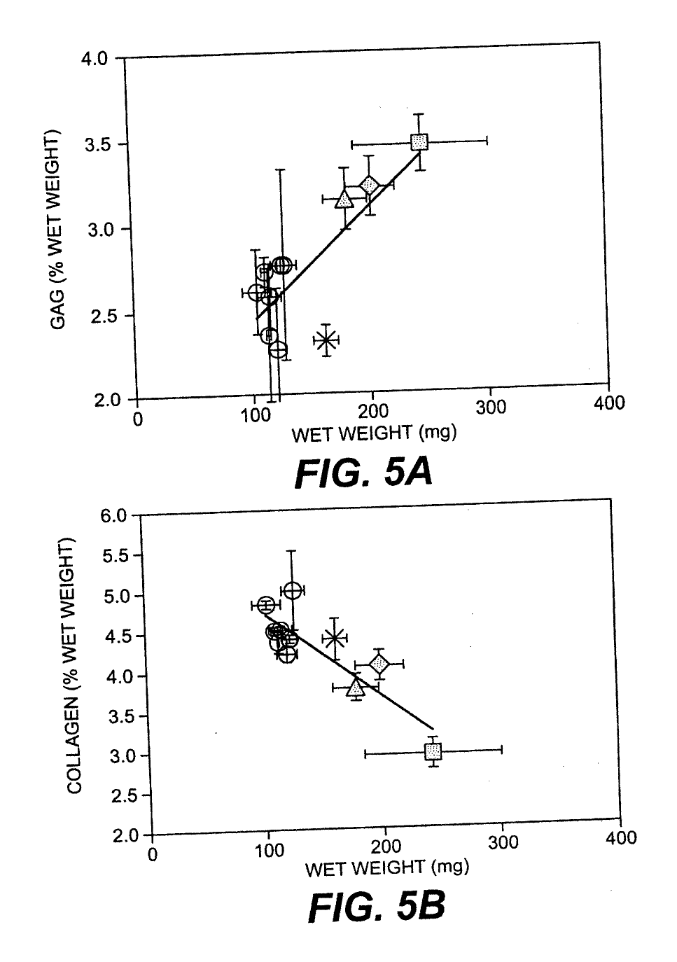

Each BMP at 100 ng/ml increased the total number of cells per construct although the overall cellularity, that is the wet weight percentage of the cells was slightly, but statistically significantly, decreased (FIG. 2). The addition of 100 ng/ml of BMP-2, BMP-12, or BMP-13 increased the weight percent of GAG in the constructs from 2.72±0.9% to 3.46±0.18%, 3.21±0.18%, and 3.13±0.19%, respectively (FIG. 3), and decreased the weight percent of collagen from 4.47±0.04% to 2.91±0.19%, 4.02±0.20%, and 3.75±0.18%, respectively (FIG. 4). At lower concentrations (1 and 10 ng/ml), the BMPs had either no or an opposite effect, slightly decreasing the weight percent of GAG (for BMP-13) and slightly increasing the weight percent of collagen (for BMP-2 and BMP-12) (FIGS. 3, 4). Over the range of conditions studied BMP-induced increases in construct weight correlated positively with the weight percent of GAG (FIG. 5A) and negatively with the weight percent of collagen (FIG. 5B). As a benchmark to which the effects of BMPs can be compared, 50 ng/ml IGF-I increased construct wet weights by 1.5-fold (FIG. 1) but did not significantly affect the weight percent of cells (FIG. 2), GAG (FIG. 3), or collagen (FIG. 4).

The foregoing descriptions detail presently preferred embodiments of the present invention. Numerous modifications and variations in practice thereof are expected to occur to those skilled in the art upon consideration of these descriptions. Those modifications and variations are believed to be encompassed within the claims appended hereto. All of the documents cited in this application are incorporated by reference in their entirety.

1. A composition for increasing the growth rate or modulating the development of engineered cartilage comprising
a) at least one active agent chosen from TGF-β superfamily proteins; and
b) at least one of biomaterial scaffold, suture, allograft, or autograft as a carrier.

2. The composition of claim 1, wherein said at least one active agent is a BMP.

3. The composition of claim 2, wherein the BMP is chosen from at least one of BMP-2, BMP-12, and BMP-13.

4. The composition of claim 1, wherein the biomaterial scaffold is polyglycolic acid.

5. The composition of claim 1, wherein the biomaterial scaffold is in a form chosen from gels, sponges, cellulose films, and meshes.

6. The composition of claim 1, wherein the composition further comprises cartilage cells.

7. The composition of claim 6, wherein the cartilage cells are chondrocytes.

8. A method for treating at least one cartilage defect comprising administering to a patient with a cartilage defect the composition of claim 1 and allowing the composition to treat the cartilage defect.

9. The method of claim 8, wherein the composition further comprises cartilage cells.

10. The method of claim 8, wherein the composition comprises at least one of BMP-2, BMP-12, and BMP-13.

11. The method of claim 8, wherein the composition is sutured into an injury site; or applied to or wrapped around cartilaginous tissue.

12. A method for increasing the growth rate or modulating the development of cartilage comprising:
adding a composition comprising
a) at least one active agent chosen from TGF-β superfamily proteins; and
b) at least one of biomaterial scaffold, suture, allograft, or autograft as a carrier
in an amount effective for increasing the growth rate or modulating the development of the cartilage.

13. The method of claim 12, wherein said at least one active agent is a BMP.

14. The method of claim 13, wherein the BMP is chosen from BMP-2, BMP-12, and BMP-13.

15. The method of claim 12, wherein the biomaterial scaffold is polyglycolic acid.

16. The method of claim 12, wherein the biomaterial scaffold is in a form chosen from gels, sponges, cellulose films, and meshes.

17. The method of claim 12, wherein the biomaterial scaffold is sutured into an injury site or wrapped around cartilaginous tissue.

18. The method of claim 12, wherein the composition is added locally as an implant or device.

19. The method of claim 12, wherein the composition is encapsulated for delivery to a site of tissue damage.
